(19) United States
(12) Patent Application Publication
(10) Pub. No.: US 2017/0007562 A1
(43) Pub. Date: Jan. 12, 2017

(54) Use of Guanidinoacetic Acid and/or Creatine for Increasing the Hatch Rate

(71) Applicant: EVONIK DEGUSSA GMBH, Essen (DE)

(72) Inventors: Meike Rademacher-Heilshorn, Wulsbüttel (DE); Ramalho Rodrigueiro, Chapecó-SC (BR)

(73) Assignee: Evonik Degussa GmbH, Essen (DE)

(21) Appl. No.: 15/117,719

(22) PCT Filed: Jan. 30, 2015
(86) PCT No.: PCT/EP2015/051895

§ 371 Date: Aug. 9, 2016

(30) Foreign Application Priority Data
Feb. 11, 2014 (EP) 14154686.1

(51) Int. Cl. A61K 31/198; A23K 10/30; A23K 20/147; A23K 20/158; A23K 20/174; A61K 9/00; A23K 20/26; A61K 36/48; A61K 36/899; A61K 33/10; A61K 33/42; A61K 33/14; A23K 50/75; A23K 20/24

(52) U.S. Cl. CPC A61K 31/198; A23K 50/75; A23K 10/30; A23K 20/147; A23K 20/158; A23K 20/174; A23K 20/24; A23K 20/26; A61K 36/48; A61K 36/899; A61K 33/10; A61K 33/42; A61K 33/14; A61K 9/0056

(57) ABSTRACT

Surprisingly, it has been found in accordance with the invention that, by adding guanidinoacetic acid, creatine and/or their salts to the feed of parent animals, the hatching rate of eggs can be increased, the embryo mortality can be reduced and the chicks' growth and feed conversion can be increased.

The present invention relates to the use of guanidinoacetic acid and/or creatine and/or their salts for increasing the hatching rate of eggs, for reducing embryo mortality, for improving chick growth and/or for improving the chicks' feed conversion.

Guanidinoacetic acid is a bodily substance which occurs in animals and also in humans and which plays a central role in creatine biosynthesis. Creatine can both be taken up with the food and be formed endogenously. The biosynthesis starts from glycine and L-arginine. In mammals, the guanidine group of L-arginine is cleaved by the enzyme amidinotransferase mainly in the kidneys, but also in the liver and in the pancreas, and an N—C—N group is transferred to the glycine. In this process, L-arginine is converted into L-ornithine. In the next step, the guanidinoacetic acid thus formed is converted into creatine with the aid of the enzyme transmethylase, a process which, in vertebrates, exclusively takes place in the liver. In this process, S-adenosylmethionine acts as methyl group donor. The creatine subsequently diffuses into the circulation, where it is transported to the target organs. The transport across the cell membrane into the cells is effected by a specific creatine transporter.

Guanidinoacetic acid is furthermore known to have antibacterial activity and has been applied successfully in animal experiments against bacterial infections (Staphylococcus aureus) (Preparation for protecting mammals against infection (Stanley Drug Products Inc., USA). Neth. Appl (1976), 7 pp. NL 7411216).

Creatine plays an important role in the cell's energy metabolism, where, besides adenosine triphosphate (ATP), it forms an essential energy reserve of the muscle in the form of energy-rich phosphocreatine. When the muscle is at rest, ATP can transfer a phosphate group to creatine, giving rise to phosphocreatine, which is then in direct equilibrium with ATP. When the muscle works, it is of crucial importance to replenish the ATP reserves as quickly as possible. Phosphocreatine becomes available for this purpose during the first seconds of maximum muscle work. This phosphocreatine is capable of transferring, through the enzyme creatine kinase, a phosphate group to adenosine diphosphate in a very rapid reaction and therefore of regenerating ATP. This is also called the Lohmann reaction.

Creatine has long been known as a suitable food supplement and animal feed. Upon hard and prolonged muscle work, the creatine reserves which are naturally present in the body are rapidly depleted. This is why targeted administrations of creatine have positive effects on stamina and performance, in particular in competitive sportsmen and women, and undesirable accumulation processes in the body or disadvantageous degradation products are unknown in this context. The reason herefor is that creatine, when supplied in unduly high quantities, is excreted by the body in the form of creatinine.

Besides creatine itself, in other words creatine monohydrate, a large number of other creatine salts, such as creatine ascorbate, creatine citrate, creatine pyruvate and others, have in the meantime also proved to be suitable food supplements. European patent EP 894 083 and the German Offenlegungsschrift DE 197 07 694 A1 may be mentioned at this point as representatives of the prior art.

Creatine and guanidinoacetic acid do not only have proven positive effects in humans, but also in animals, which is why their use in animal feeds has likewise already been described. Thus, it is known from international patent applications WO 00/67 590 and WO 05/120246 to use creatine and/or guanidinoacetic acid and their salts as feed additive for breeding animals and fatteners, for increasing fertility and reproductive performance, as a replacement for meat meal, fish meal and/or antimicrobial growth promoters, growth hormones and anabolic steroids.

GB 2 300 103 teaches the use of creatine in the form of a dog biscuit, for which purpose the creatine monohydrate together with meat is fed as an extruded substance. Owing to its poor solubility, creatine monohydrate's bioavailability is only limited, and so it is recommended that it be used together with other physiologically active compounds, preferably in salt form. German Offenlegungsschrift DE 198 36 450 A1 relates to the use of stable pyruvic acid salts, in particular creatine pyruvate, in formulations which are suitable for animal nutrition.

DE 100 03 835 A1 relates to formulations for use in states of dehydration as are generally observed in older people, in particular those with limited mobility. In this case, creatine acts as transport medium for water so as to supply moisture to those tissues which are most affected by dehydration symptoms.

Surprisingly, it has now been found in accordance with the invention that guanidinoacetic acid has an effect on the hatching rate of eggs. Indeed, guanidinoacetic acid results in a significantly increased hatching rate. This is especially surprising in particular because it has been previously described in the prior art that creatine had no effect on the hatching rate (Halle et al, Landbauforschung Völkenrode (2006), 56 (1/2), 11-18).

Furthermore, it has been found that feeding guanidinoacetic acid to the parent animals also results in a reduced embryo mortality, in an improved chick growth, in particular in an improved weight gain, and in an improved chicks' feed conversion, even if the chicks themselves are not fed guanidinoacetic acid.

A first subject-matter of the present invention is, therefore, the use of a compound selected among guanidinoacetic acid, creatine and salts of these molecules and mixtures thereof for increasing the hatching rate in eggs and/or for reducing embryo mortality and/or for improving chick growth and/or for improving the chicks' feed conversion.

Preferred subject-matter in this context is the use of guanidinoacetic acid and/or its salts for increasing the hatching rate in eggs and/or for reducing embryo mortality and/or for improving chick growth and/or for improving the chicks' feed conversion.

Especially preferred in this context is the use of guanidinoacetic acid and/or its salts for increasing the hatching rate in eggs.

A further subject-matter of the present invention is therefore likewise a method for increasing the hatching rate in eggs and/or for reducing embryo mortality and/or for improving chick growth and/or for improving the chicks' feed conversion, in which method the birds, preferably parent animals, are fed a feed which comprises a compound, selected among guanidinoacetic acid, creatine and their salts and mixtures thereof so as to increase the hatching rate, to reduce embryo mortality, to increase growth and/or improve feed conversion.

Preferred subject-matter in this context is a method for increasing the hatching rate in eggs and/or for reducing embryo mortality and/or for improving chick growth and/or for improving the chicks' feed conversion, in which method the birds, preferably parent animals, are fed a feed which comprises guanidinoacetic acid or its salts so as to increase the hatching rate, to reduce embryo mortality and/or to increase growth.

Especially preferred in this context is a method of increasing the hatching rate in eggs, in which method the birds, preferably parent animals, are fed a feed which comprises guanidinoacetic acid or its salts so as to increase the hatching rate.

According to the invention, “improving chick growth” is understood in particular as meaning an improvement of the chicks' weight gain.

Uses and methods according to the invention are implemented in a preferred embodiment for non-therapeutic purposes.

The hatching rate plays an important role in particular in the production of productive poultry. In this context, it is decisive that a sufficient amount of freshly hatched chicks can be provided. Besides the fertility of the parent animals employed, the hatching rate of the eggs is one of the decisive factors in this context for providing the chicks. In this respect, increasing the hatching rate is a key improvement of the method's economicalness.

The eggs are preferably eggs of any type of bird and/or poultry. The eggs may in particular be eggs of productive or domestic poultry, but also eggs of fancy poultry or wild fowl. The eggs are especially preferably hatchery eggs.

Preferred productive poultry in this context are chickens, turkeys, ducks and geese. The productive livestock in this context is preferably poultry optimized for producing young stock. This type of poultry is also referred to as parent animals. Preferred parent animals are, accordingly, parent broilers, parent ducks, parent turkeys and parent geese. The eggs of these parent animals are referred to as hatchery eggs.

Preferred fancy poultry or wild fowl are peacocks, pheasants, partridges, guinea fowl, quails, capercaillies, grouse, pigeons and swans, with quails being especially preferred.

Further preferred poultry are ostriches and parrots.

In accordance with the invention, guanidinoacetic acid, creatine and their salts can be employed in a wide dosage range. Daily doses are, for example, in the range of between approximately 5 mg and approximately 1200 mg, in particular in the range of from approximately 10 mg to approximately 600 mg, preferably in the range of from approximately 25 to approximately 300 mg, per kilogram live weight, in particular in the case of parent broilers. Individual doses are, generally, in the range of between 5 mg to approximately 600 mg, preferably in the range of from approximately 10 to approximately 150 mg.

In the light of the described use as feed additive, suitable doses, depending on the poultry species, are preferably of from approximately 0.01 to approximately 100 g/kg of feeding stuff, with amounts of from approximately 0.4 to approximately 2.0 g/kg of feeding stuff—that is to say 0.04 to 0.20% by weight—being considered as especially preferred. It is very especially preferred to employ amounts of from 0.07 to 0.18% by weight, in particular from 0.08 to 0.16% by weight.

Owing to feeding feed comprising guanidinoacetic acid, especially high hatching rates were observed in quails.

A further subject-matter of the present invention is therefore a method for feeding wild fowl, characterized in that the wild fowl is fed a feed which comprises a compound selected from among guanidinoacetic acid, creatine and their salts and mixtures of these, preferably guanidinoacetic acid and/or its salts.

In this context, the wild fowl is preferably selected among quails, pigeons, guinea fowl, partridges, peacocks, capercaillies, grouse and pheasants, especially preferably quails.

Therefore, a further subject-matter of the present invention is also a method of feeding small birds, characterized in that they are fed a feed which comprises a compound selected among guanidinoacetic acid, creatine and their salts and mixtures thereof, preferably guanidinoacetic acid and/or its salts.

In accordance with the invention, a small bird is taken to mean a bird which, when fully grown, reaches a weight of on average not more than 500 grams, preferably not more than 400 or 300 grams, in particular 20 to 300 grams, especially preferably 50 to 200 grams. Feeding guanidinoacetic acid to small birds has not been described as yet in the prior art.

In the methods for feeding wild fowl and/or small birds, the feed comprises guanidinoacetic acid and/or creatine and/or their salts, especially preferably guanidinoacetic acid and/or its salts, preferably in an amount of from 0.06 to 0.2% by weight, in particular in an amount of from 0.1 to 0.2% by weight, especially preferably in an amount of from 0.12 to 0.18% by weight, above all in an amount of from 0.14 to 0.16% by weight.

In the case of parent broilers, smaller dosages have proved to be more advantageous. Thus, the optimal dosage in the case of parent broilers is 0.06 to 0.14% by weight, in particular 0.07 to 0.13% by weight, above all 0.08 to 0.12% by weight.

The birds may be fed guanidinoacetic acid and/or creatine and/or their salts over the entire lifetime of the poultry in order to achieve the effects according to the invention. In this context, feeding preferably takes place after sexual maturity has been reached, i.e. as soon as the poultry is capable of laying eggs. In the case of parent broilers, this is the 23rd to 24th week of their life.

It must furthermore be taken into consideration that, in the case of parent animals, the hatching rate will naturally go through a maximum. Accordingly, a maximum—in absolute terms—increase in the hatching rate can be achieved before or after going through this maximum. In nature, the maximum hatching rates will, in the case of parent broilers, be gone through during the 35th to 37th week of their lives. Therefore, feeding the poultry guanidinoacetic acid and/or creatine and/or their salts makes it possible in particular to increase the hatching rate of sexually mature parent animals which are either not yet capable of producing eggs with an optimal natural hatching rate and/or which have already exceeded the age (in particular from approximately the 40th week of their lives) of producing eggs with an optimal natural hatching rate.

However, feeding guanidinoacetic acid and/or creatine and/or their salts also allows to naturally increase the hatching rate of eggs in the case of parent animals which, in terms of the hatching rate, are naturally already in the maximum range. This is because even the naturally achievable optimum is markedly below 100%.

In accordance with the invention, the guanidinoacetic acid and/or the creatine and/or their salts can be provided, for example, as a powder, as granules, as lozenges, as capsules, as pellets or as jelly (hydrocolloidal) products for incorporation into the feeding stuff. Depending on the respective specific intended use, it is preferred to employ the guanidinoacetic acid and/or the creatine and/or their salts as feed additive in combination with other physiologically active substances, with, in particular, carbohydrates, fats, amino acids (for example creatine), proteins, vitamins, minerals, trace elements and their derivatives and any mixtures of these being especially suitable. Preference is given to methionine, betaine and choline and to other physiologically effective methyl group donors. Betaine and choline can be converted into methionine in the body in the presence of homocysteine, which plays a role in particular in the synthesis of creatine starting from guanidinoacetic acid. Methyl groups, which are transferred with formation of homocysteine from S-adenosylmethionine are required for this purpose. If not enough betaine or choline is available, methionine is consumed, which may result in a methionine deficit in the metabolism.

In a method which is preferred in accordance with the invention, the feed employed for feeding the birds, in particular parent animals, comprises at least one, preferably at least two, three or four, in particular at least five, six or seven, especially preferably at least eight, of the following components:
a) maize or maize meal, preferably in an amount of from 20 to 70% by weight, in particular 30 to 60% by weight;
b) extracted soybean meal, preferably in an amount of from 10 to 50% by weight, in particular 15 to 45% by weight;
c) an oil or fat component, in particular soya oil, preferably in an amount of from 1 to 10% by weight, in particular 3 to 8% by weight;
d) calcium carbonate, preferably in an amount of from 4 to 14% by weight, in particular 6 to 12% by weight;
e) dicalcium phosphate, preferably in an amount of from 0.2 to 2.5% by weight, in particular 0.5 to 1.5% by weight;
f) wheat or wheat flour, preferably in an amount of up to 60% by weight, in particular up to 40 or 20% by weight;
g) barley or barley flour, preferably in an amount of up to 40% by weight, in particular up to 20 or 10% by weight;
h) amino acids, preferably selected among lysine, methionine, threonine, valine and arginine, and mixtures of these;
i) vitamins.

In an embodiment which is preferred in accordance with the invention, the guanidinoacetic acid, the creatine and/or their salts as feed additive are employed in predominantly vegetarian rations.

The expression “predominantly vegetarian ration” used herein describes a ration which, preferably in agreement with the legal guidelines of the European Union, does not comprise any animal components. The exception of this context is only a possible addition of fish meal. Furthermore, a “predominantly vegetarian ration” in accordance with the present invention is also understood as meaning a partial substitution of fish meal or meat meal by guanidinoacetic acid, creatine and/or their salts. Alternatively, however, it is also possible to employ guanidinoacetic acid, creatine and/or their salts in addition to animal components, in particular in combination with fish meal and/or meat meal.

A feeding stuff comprising the components which follow has proved to be especially advantageous in accordance with the invention:
a) crude protein in an amount of from 10 to 30% by weight, in particular of from 12 to 25% by weight,
b) calcium in an amount of from 2 to 5% by weight,
c) phosphorus in an amount of from 0.2 to 0.6% by weight,
d) methionine and/or cysteine in an amount of from 0.25 to 1.0% by weight,
e) lysine in an amount of from 0.5 to 1.5% by weight,
f) guanidinoacetic acid in an amount of from 0.04 to 0.20% by weight, preferably 0.07 to 0.18% by weight, especially preferably 0.08 to 0.16% by weight.

Therefore, this feeding stuff, too, is subject-matter of the present invention, as are methods for feeding birds, in particular parent animals, in which this feed is employed.

In accordance with the invention, it is preferred to employ in particular a feeding stuff which comprises the following components:
a) maize or maize meal in an amount of from 20 to 70% by weight, preferably 30 to 60% by weight;
b) extracted soybean meal in an amount of from 10 to 50% by weight, preferably 15 to 45% by weight;
c) guanidinoacetic acid and/or creatine and/or a salt of these in an amount of from 0.02 to 0.2% by weight, preferably 0.07 to 0.18% by weight, in particular 0.08 to 0.16% by weight;
d) optionally an oil or fat component, in particular soya oil, preferably in an amount of from 1 to 10% by weight, in particular 3 to 8% by weight;
e) optionally calcium carbonate, preferably in an amount of from 4 to 14% by weight, in particular 6 to 12% by weight;
f) optionally dicalcium phosphate, preferably in an amount of from 0.2 to 2.5% by weight, in particular 0.5 to 1.5% by weight;
g) optionally wheat or wheat flour, preferably in an amount of up to 60% by weight, in particular up to 40 or 20% by weight;
h) optionally barley or barley flour, preferably in an amount of up to 40% by weight, in particular up to 20 or 10% by weight;
i) optionally amino acids, preferably selected among lysine, methionine, threonine, valine and arginine and mixtures of these;
j) optionally vitamins.

This feeding stuff, too, is therefore subject-matter of the present invention, as is a method in which this feed is employed for feeding birds, in particular parent animals.

Guanidinoacetic acid can be prepared in a simple and economical fashion, for example by processes like reacting glycine and cyanamide in aqueous solutions (Production of guanidino fatty acids (Vassel, Bruno; Janssens, Walter D.) (1952), U.S. Pat. No. 2,620,354 ; Method of preparation of guanidino fatty acids (Vassel, Bruno; Garst, Roger) (1953), 5 pp. U.S. Pat. No. 2,654,779).

In contrast to creatine and creatine monohydrate, guanidinoacetic acid and its salts are, moreover, markedly more stable in the acidic aqueous solution, and they are converted into creatine only under physiological conditions. In this context, guanidinoacetic acid is only converted into creatine after its absorption, above all in the liver. In contrast to creatine, therefore, the predominant fraction of the administered, or fed, compounds, guanidinoacetic acid and/or salts of guanidinoacetic acid, is not degraded by instability reactions, for example in the stomach, and secreted before the absorption, but is indeed available when the corresponding physiological metabolic reaction takes place.

Suitable for the purposes of the present invention are, in principle, all those guanidinoacetic acid salts and creatine salts which are nutritionally acceptable. Compounds which have proven to be particularly advantageous for the use according to the invention are guanidinoacetic acid salts and creatine salts which are obtained with hydrochloric acid, hydrobromic acid and phosphoric acid. It is also possible to employ mixtures of guanidinoacetic acid and/or creatine together with one or more of these salts, or else mixtures of the salts with each other.

In total, the present invention serves to provide guanidinoacetic acid, creatine and their salts for novel uses as feeding stuff or feeding stuff additive in poultry nutrition. The examples which follow illustrate the present invention further.

### WORKING EXAMPLES

### Example 1

### Effect of Quanidinoacetic Acid on the Hatching Rate of Quail Eggs

240 parent quails aged 25 weeks were randomly assigned to 5 different rations. Each ration had in each case 8 cages assigned to it, each of which contained a total of 6 birds (4 females, 2 males). Each of the cages was exposed to 17 hours light (natural and artificial), followed by 7 hours dark.

The basic feed employed was a mixture which consisted essentially of maize meal and extracted soybean meal (see table 1). Guanidinoacetic acid (GAA) was added to the basic feed in 4 different amounts (0.06; 0.12; 0.18; 0.24% by weight) giving in total 5 different feed mixtures—with the basic feed without guanidinoacetic acid as reference. The inert constituents of the basic feed were in each case partially substituted by the added guanidinoacetic acid. Guanidinoacetic acid was employed in the form of the commercial product CreAMINO® (96% GAA, Evonik Industries, Germany). During the experiment, the feed was administered ad lib in the form of a meal. Furthermore, the birds were also supplied with sufficient water.

TABLE 1
Basic feed for feeding the parent animals and the hatched chicks

| | | Chicks | |
|---|---|---|---|
| | Parent animals | 1 to 21 | 22 to 35 |
| | 25 to 29 weeks | days | days |
| Constituents (% by weight) | | | |
| Maize | 45.20 | 47.74 | 58.73 |
| Extracted soybean meal (45%) | 39.20 | 45.88 | 35.35 |
| Soya oil | 4.100 | 2.530 | 2.890 |
| Dicalcium phosphate | 1.150 | 1.030 | 0.850 |
| Calcium carbonate | 8.600 | 1.320 | 1.080 |
| Salt | 0.400 | 0.400 | 0.400 |
| Vitamin and mineral premix | 0.400 | 0.400 | 0.400 |
| Inert constituents | 0.300 | — | — |
| DL-Methionine | 0.189 | 0.391 | 0.235 |
| L-Lysine HCl | 0.045 | 0.130 | 0.000 |
| L-Threonine | 0.073 | 0.195 | 0.068 |
| L-Valine | 0.135 | — | — |
| L-Arginine | 0.093 | — | — |
| Total | 100.00 | 100.00 | 100.00 |
| Nutrients (% by weight) | | | |
| Crude protein | 22.00 | 25.00 | 21.00 |
| Calcium | 3.500 | 0.850 | 0.700 |
| Digestible phosphorus | 0.320 | 0.320 | 0.270 |
| Potassium | 0.814 | 0.978 | 0.817 |
| Sodium | 0.170 | 0.177 | 0.178 |
| Chlorine | 0.314 | 0.314 | 0.244 |
| Lysine (digestible) | 1.130 | 1.370 | 1.020 |
| Met + Cys (digestible) | 0.770 | 1.040 | 0.800 |
| Threonine (digestible) | 0.810 | 1.014 | 0.780 |
| Tryptophan (digestible) | 0.290 | 0.290 | 0.234 |
| Arginine (digestible) | 1.510 | 1.616 | 1.320 |
| Valine (digestible) | 1.050 | 1.061 | 0.890 |

When the quails had reached an age of 29 weeks, the eggs laid were collected on seven successive days and stored for six days in a room at a temperature of 20° C. Thereafter, in each case 196 eggs per ration were incubated in an automatic hatcher. After incubation for 15 days in the hatcher, the eggs were transferred into a hatching room with a temperature of 37° C. and relative atmospheric humidity of 65%. After two days in the hatching room, the hatching rate was determined as the ratio of hatched quails to the number of incubated eggs. Equally, the total fertility was determined as the ratio of fertilized eggs to the total number of incubated eggs. Furthermore, the hatching rate of fertilized eggs was determined as the ratio of hatched quails to the number of fertilized eggs. Furthermore, the embryo mortality, too, was determined. Finally, the guanidinoacetic acid, creatine and creatinine contents in the obtained eggs were determined.

A significant effect of guanidinoacetic acid on the hatching rate of the eggs, in particular the fertilized eggs, was identified. In addition, the mortality rate of the hatched chicks was markedly reduced (see Table 2). Furthermore, it can be seen that it was possible to significantly increase the observed effects from a dosage of above 0.1% by weight GAA. Using square regression analysis, the optimum value for GAA supplementation in terms of the total reproduction was identified as being 0.14% by weight GAA, in terms of the hatching rate 0.15% by weight, in terms of the hatching rate of fertilized eggs 0.16% by weight and in terms of the mortality rate 0.16% by weight, so that a range of from 0.12-0.18% by weight GAA can be specified as the optimum of feeding the parent animals.

TABLE 2
Effect of GAA on the total fertility, the hatching rate
and the embryo mortality in quails

| | | | Hatching | Hatching rate |
|---|---|---|---|---|
| | Total fertility | Mortality | rate | of the fertilized eggs |
| GAA | (%) | (%) | (%) | (%) |
| 0.00% | 92.38 ± 2.51 | 18.96 ± 2.62 | 73.41 ± 4.25 | 79.26 ± 3.06 |
| 0.06% | 95.27 ± 1.81 | 18.75 ± 2.76 | 76.52 ± 3.78 | 80.19 ± 3.07 |
| 0.12% | 97.05 ± 1.51 | 6.40 ± 1.96 | 90.69 ± 2.10 | 93.49 ± 2.04 |
| 0.18% | 98.32 ± 0.82 | 10.92 ± 3.32 | 87.39 ± 3.20 | 88.94 ± 3.33 |
| 0.24% | 94.02 ± 1.29 | 12.64 ± 1.89 | 81.38 ± 2.50 | 86.51 ± 2.03 |

Likewise, it has been found that feeding the parent animals GAA is also reflected in the GAA, creatine and creatinine contents in the eggs (Table 3). The higher the GAA content in the feed, the higher the found content of GAA and its metabolites creatine and creatinine.

TABLE 3
Effect of GAA in the parent animals' feed on the GAA,
creatine and creatinine contents in the eggs

| | GAA | Creatine | Creatinine |
|---|---|---|---|
| GAA | μ/g egg | | |
| 0.00% | 0.00 ± 0.00 | 19.63 ± 1.15 | 2.25 ± 0.16 |
| 0.06% | 0.25 ± 0.25 | 24.88 ± 0.91 | 2.88 ± 0.23 |
| 0.12% | 1.50 ± 0.19 | 24.13 ± 1.06 | 2.63 ± 0.18 |
| 0.18% | 1.88 ± 0.29 | 25.38 ± 0.91 | 2.88 ± 0.23 |
| 0.24% | 3.00 ± 0.46 | 26.75 ± 1.52 | 3.25 ± 0.16 |

Finally, the effect of GAA feeding of the parent animals on the properties of the hatched chicks was also studied. To this end, in each case 80 chicks which had been obtained subsequently to feeding the parent animals with the respective ratios were subjected to a two-step feeding programme. Here, the chicks were subjected to a GAA-free ration. Here, all chicks were given the same feed. The feeding stuff of the chicks for the first three weeks and for weeks four to five can be seen from Table 1. The data determined were chick weight on the day of hatching, chick performance (feed intake, weight increase and feed conversion) and creatine content in the muscle tissue of the freshly hatched chicks.

In this context, it was found above all that owing to the parent animals having been fed GAA, the creatine content in the muscle tissue of the freshly hatched chicks and, likewise, the chicks' weight gain and feed conversion were markedly improved (Table 4).

TABLE 4
Effect on GAA feeding of the parent animals on
the properties of the hatched chicks

| | | | | | Feed |
|---|---|---|---|---|---|
| | Weight on | Creatine in the | Weight gain | Feed intake | conversion |
| GAA | day 1 (g) | muscle (mg/g)¹ | (g) | (g/day) | (g/g) |
| 0.00% | 9.62 ± 0.18 | 0.435 ± 0.015 | 199.45 ± 10.2 | 755 ± 6.83 | 3.789 ± 0.180 |
| 0.06% | 9.60 ± 0.16 | 0.541 ± 0.026 | 230.74 ± 7.6 | 748 ± 11.07 | 3.248 ± 0.063 |
| 0.12% | 9.74 ± 0.31 | 0.489 ± 0.058 | 224.98 ± 2.9 | 768 ± 11.08 | 3.414 ± 0.048 |
| 0.18% | 9.65 ± 0.16 | 0.481 ± 0.040 | 217.22 ± 0.6 | 718 ± 8.95 | 3.305 ± 0.033 |
| 0.24% | 9.41 ± 0.17 | 0.433 ± 0.033 | 222.86 ± 1.9 | 770 ± 26.13 | 3.455 ± 0.090 |

### Example 2

### Effect of Guanidinoacetic Acid on the Hatching Rate of Hatchery Eggs of Parent Broilers

120 parent broilers (Cobb 500) were randomly assigned to 5 different 10-week feeding measures from week 50 to week 60 of their lives. In each case 6 cages, each of which contained a total of 4 parent hens were subjected to each feeding measure. The basic feed employed was a mixture consisting essentially of maize meal and soybean meal (Table 5). Guanidinoacetic acid (GAA) was added to the basic feed in 4 different amounts (0.06; 0.12; 0.18; 0.24% by weight) so that the total 5 different feed mixtures—with the basic feed without guanidinoacetic acid as reference—were obtained. In this context, in each case the inert constituents of the basic feed were substituted to some extent by the added guanidinoacetic acid. Guanidinoacetic acid was employed in the form of the commercial product CreAMINO® (96% GAA, Evonik Industries, Germany). The parent animals were given the feed meal as per instructions of the Cobb Breeder Management Guide. In week 60 of their lives, the hens were twice artificially inseminated, the second insemination taking place 4 days after the first one. The semen used for the insemination was obtained from cockerels which had not been fed GAA. The semen was obtained by using abdominal massage. The semen obtained was collected and the sperm cell concentration was determined in order to ensure that all hens were inseminated with the same number and the same volume of sperm cells. After carrying out the insemination, the laid eggs were collected on 10 successive days. The eggs obtained were finally incubated under identical conditions, and the egg production rate, fertility and hatching rate of the fertilized eggs were subsequently determined.

TABLE 5
Basic feed for feeding the parent animals

| | % by weight |
|---|---|
| Constituent | |
| Maize | 51.55 |
| Wheat | 6.00 |
| Extracted soybean meal (44%) | 26.19 |
| Soya oil | 5.07 |
| Calcium carbonate | 8.07 |
| Dicalcium phosphate | 1.53 |
| Salt | 0.38 |
| L-Lysine HCl | 0.04 |
| DL-Methionine | 0.22 |
| L-Threonine | 0.05 |
| Vitamin/mineral premix | 0.40 |
| Inert constituents | 0.50 |
| Total | 100.00 |
| Nutrients (% by weight) | |
| Crude protein | 15.50 |
| Calcium | 3.50 |
| Phosphorus (digestible) | 0.42 |
| Met + Cys (digestible) | 0.60 |
| Lysine (digestible) | 0.80 |

A significant effect of guanidinoacetic acid on fertility and the hatching rate of the eggs was found (Table 6). It can furthermore be seen that the best results were obtained with a dosage of from 0.08 to 0.12% by weight GAA. The optimum dosage rate was therefore somewhat lower than in the case of quail feeding.

TABLE 6
Effect of GAA on egg production, fertility and
the hatching rate of fertilized eggs

| | Guanidinoacetic acid, % by weight | | | | |
|---|---|---|---|---|---|
| Parameter | 0.00 | 0.04 | 0.08 | 0.12 | 0.16 |
| Egg production % | 52.12 | 50.42 | 53.18 | 53.44 | 50.50 |
| Fertility % | 80.27 | 85.15 | 96.51 | 96.43 | 81.76 |
| Hatching rate % | 65.66 | 65.59 | 83.76 | 83.95 | 65.04 |

1-15. (canceled)

16. A method for increasing the hatching rate in eggs, and/or for reducing embryo mortality, and/or for improving chick growth, and/or for improving the chicks' feed conversion, comprising feeding parent animals a feed which comprises guanidinoacetic acid; creatine; salts of these compounds; and mixtures of the compounds and/or their salts.

17. The method of claim 16, wherein said parent animals are selected from the group consisting of: poultry; broilers; geese; ducks; turkeys; quails; peacocks; pheasants; pigeons; guinea fowl; partridges; grouse; and ostriches.

18. The method of claim 17, wherein said parent animals are selected from the group consisting of: chickens; broilers and quails.

19. The method of claim 16, wherein said guanidinoacetic acid, and/or creatine, and/or salts of these compounds are present in the feed in an amount of from 0.04 to 0.20% by weight.

20. The method of claim 19, wherein guanidinoacetic acid and/or creatine and/or salts of these compounds are present in the feed in an amount of from 0.08 to 0.16% by weight.

21. The method of claim 16, wherein said feed further comprises at least one of the following components:
a) maize or maize meal, in an amount of from 20 to 70% by weight;
b) extracted soybean meal, in an amount of from 10 to 50% by weight;
c) an oil or fat, in an amount of from 1 to 10% by weight;
d) calcium carbonate, in an amount of from 4 to 14% by weight;
e) dicalcium phosphate, in an amount of from 0.2 to 2.5% by weight;
f) wheat or wheat flour, in an amount of up to 60% by weight;
g) barley or barley flour, in an amount of up to 40% by weight;
h) one or more amino acids selected from the group consisting of: lysine; methionine; threonine; valine; arginine; and mixtures thereof;
i) vitamins.

22. The method of claim 21, wherein said feed further comprises at least three of components a)-i).

23. The method of claim 16, wherein said parent animals are wild fowl, and said feed comprises guanidinoacetic acid, and/or creatine, and/or salts of these compounds in an amount of from 0.06 to 0.2% by weight.

24. The method of claim 23, wherein said feed further comprises at least one of the following components:
a) maize or maize meal, in an amount of from 20 to 70% by weight;
b) extracted soybean meal, in an amount of from 10 to 50% by weight;
c) an oil or fat, in an amount of from 1 to 10% by weight;
d) calcium carbonate, in an amount of from 4 to 14% by weight;
e) dicalcium phosphate, in an amount of from 0.2 to 2.5% by weight;
f) wheat or wheat flour, in an amount of up to 60% by weight;
g) barley or barley flour, in an amount of up to 40% by weight;
h) one or more amino acids selected from the group consisting of: lysine; methionine; threonine; valine; arginine; and mixtures of thereof;
i) vitamins.

25. The method of claim 16, wherein said parent animals are quail, and said feed comprises guanidinoacetic acid, and/or creatine, and/or salts of these compounds in an amount of from 0.06 to 0.2% by weight.

26. The method of claim 25, wherein said feed further comprises at least one of the following components:
a) maize or maize meal, in an amount of from 20 to 70% by weight;
b) extracted soybean meal, in an amount of from 10 to 50% by weight;
c) an oil or fat component, in an amount of from 1 to 10% by weight;
d) calcium carbonate, in an amount of from 4 to 14% by weight;
e) dicalcium phosphate, in an amount of from 0.2 to 2.5% by weight;
f) wheat or wheat flour, in an amount of up to 60% by weight;
g) barley or barley flour, in an amount of up to 40% by weight;
h) one or more amino acids selected from the group consisting of: lysine; methionine; threonine; valine; arginine; and mixtures of thereof;
i) vitamins.

27. An animal feed, comprising guanidinoacetic acid, and/or creatine, and/or a salt of these compounds in an amount of from 0.02 to 0.2% by weight.

28. The animal feed of claim 27, further comprising one or more of the following components:
a) maize or maize meal, in an amount of from 20 to 70%;
b) extracted soybean meal, in an amount of from 10 to 50%;
c) an oil or fat component, in an amount of from 1 to 10% by weight;
d) calcium carbonate, in an amount of from 4 to 14% by weight;
e) dicalcium phosphate, in an amount of from 0.2 to 2.5% by weight;
f) wheat or wheat flour, in an amount of up to 40% by weight;
g) barley or barley flour, in an amount of up to 40% by weight;
h) one or more amino acids, selected from the group consisting of: lysine, methionine, threonine, valine and arginine, and mixtures of these;
i) vitamins.

29. The animal feed of claim 28, wherein said feed comprises maize or maize meal in an amount of from 20 to 70% by weight.

30. The animal feed of claim 29, wherein said feed comprises extracted soybean meal in an amount of from 10 to 50%

31. The animal feed of claim 30, wherein said feed comprises at least 3 of components c)-i).

32. The animal feed of claim 27, wherein said guanidinoacetic acid, and/or creatine, and/or salt of these compounds is present in an amount of from 0.07 to 0.16% by weight, and further comprising:
a) maize or maize meal, in an amount of from 30 to 60% by weight;
b) extracted soybean meal, in an amount of from 15 to 45% by weight and at least three further components, selected from the group consisting of;
c) soya oil, in an amount of from 1 to 10% by weight;
d) calcium carbonate, in an amount of from 6 to 12% by weight;
e) dicalcium phosphate, in an amount of from 0.5 to 1.5% by weight;
f) wheat or wheat flour, in an amount of up to 10% by weight;
g) barley or barley flour, in an amount of up to 10% by weight;
h) one or more amino acids selected from the group consisting of: lysine; methionine; threonine; valine; arginine; and mixtures thereof; and
i) vitamins.

33. The animal feed of claim 27, further comprising one or more of the following:
a) crude protein, in an amount of from 10 to 30% by weight;
b) calcium, in an amount of from 1 to 5% by weight;
c) phosphorus, in an amount of from 0.2 to 0.6% by weight;
d) methionine and/or cysteine, in an amount of from 0.25 to 1.0% by weight;
e) lysine, in an amount of from 0.5 to 1.5% by weight.

34. The animal feed of claim 33, further comprising at least 4 of components a)-e).

35. The animal feed of claim 27, further comprising:
a) crude protein in an amount of from 12 to 25% by weight;
b) calcium in an amount of from 1 to 5% by weight;
c) phosphorus in an amount of from 0.2 to 0.6% by weight;
d) methionine and/or cysteine in an amount of from 0.25 to 1.0% by weight; and
e) lysine in an amount of from 0.5 to 1.5% by weight.
